(19) United States
(12) Patent Application Publication
(10) Pub. No.: US 2011/0136847 A1
(43) Pub. Date: Jun. 9, 2011

(54) High Concentration Formulations of Opioids and Opioid Derivatives

(76) Inventors: Tai Wah Chan, Palo Alto, CA (US); Wilma Tamraz, San Jose, CA (US); Min Lee, Saratoga, CA (US)

(21) Appl. No.: 12/885,345

(22) Filed: Sep. 17, 2010

Related U.S. Application Data
(63) Continuation of application No. 10/305,252, filed on Nov. 25, 2002, now abandoned.

(51) Int. Cl. A61K 31/46; A61K 31/4535; A61K 31/445; A61P 29/00

(52) U.S. Cl. 514/282; 514/326; 514/329

(57) ABSTRACT

The present invention provides opioid formulations suitable for long-term delivery to a subject. The formulation of the invention comprises an opioid or opioid derivative (e.g., morphine, hydromorphone, fentanyl or a fentanyl congener), and an aqueous solvent comprising a low molecular weight carboxylic acid (e.g., C₂₋₄, C₂₋₇). The invention thus provides for formulations comprising morphine, hydromorphone, fentanyl or fentanyl congeners in concentrations significantly in excess of conventional aqueous formulations, e.g., on the order about 2-fold to about 10,000-fold greater than conventional formulations, e.g., currently commercially available formulations.

### FIELD OF THE INVENTION

The invention relates to high-concentration aqueous formulations of opioids to alleviate pain.

### BACKGROUND OF THE INVENTION

Opiates in various forms, including opium, heroin and morphine, which derive from the opium poppy, have very powerful analgesic properties and have seen widespread use for anesthesia as well the treatment of pain, especially where the pain is very severe. In addition to these natural opiates, many synthetic opioids have since been synthesized including hydromorphone, fentanyl and congeners of fentanyl such as sufentanil, alfentanil, lofentanil, carfentanil, remifentanil, etc., which are many times more potent than morphine.

While the most commonly used dosage form is orally administered morphine, opioids can also be delivered by intravenous infusion (see, e.g., Scholz et al. (1996) Clin. Pharmacokinet. 31:275-92; White (1989) Anesth. Analg. 68:161-171), oral administration, (see, e.g., U.S. Pat. Nos. 4,769,372; 5,202,128; and 5,378,474), epidural or intrathecal administration (see, e.g., Vercauteren et al. (1998) Anaesthesia 53:1022-7; Stephens (1997) Am. Pam. Physician 56:463-70), transdermal application (e.g., using a transdermal patch (see, e.g., U.S. Pat. No. 4,588,580)), or subcutaneous injection (see, e.g., Paix et al. (1995) Pain 63:263-9; Bruera et al. (1988) Cancer 62:407-11; Moulin et al. (1992) Can. Med. Assoc. J. 146:891-7). For a review, see, e.g., Clotz et al. (1991) Clin. Pharm. 10:581-93; and Anderson et al. (1998) J. Pharm. Care Pain Symptom Control 6:5-21.

Unfortunately, oral administration with several disadvantages. Many extremely ill patients can no longer take drugs orally for a variety of reasons, such as the inability to swallow or gastrointestinal obstruction. Furthermore, long-term oral administration often necessitates the ingestion of multiple pills or tablets many times a day, a dosing scheme commonly associated with poor compliance. For these and other reasons, parenteral administration of opioids can be a preferred alternative to oral administration.

Parenteral administration of opioids also meets with several challenges. Many patients, especially those with chronic pain or diseases, require long-term treatment with opioids, e.g., for days, months, years and sometimes for the lifetime of the patient, and therefore require large quantities of drug to be administered over time. Also, many patients with severe pain require high doses of opioids to control pain, oftentimes with escalating requirements due to progression of the underlying disease state or development of tolerance to the opioid. Furthermore, in order to provide convenient, long-term or high-dosage pain treatment, opioids may need to be administered continuously and for a long duration. In order to provide acceptable convenience and mobility to patients, drug delivery devices must be limited in size, which in turn limits the volume of drug formulation that can be contained within the reservoir of the device. When opioids are administered for a long period using conventional formulations of opioids, the limited size of the drug reservoir of the pumps requires frequently refilling of the device, or exchanging the device for a new, filled device. Besides being inconvenient, refilling and/or exchange of an implanted drug delivery device can require the attention of a skilled health care worker and can expose the patient to possible infection.

Besides limitations imposed by device size, the absorption capacity of the tissue into which the drug formulation is infused can limit the volume amount of drug formulation that can be absorbed. For example, the absorptive capacity of the subcutaneous tissue is generally a maximum of 10 ml per hour (see e.g., Anderson et al., supra). Furthermore, infusions of large amounts of fluid into certain tissue can cause tissue edema, which causes discomfort to the patient.

Currently available commercial opioid formulations are too dilute to meet the needs of patients requiring long-term treatment or large drug doses to control pain. For example, hydromorphone hydrochloride (Dilaudid®) is currently available in an aqueous solution at a concentration of 10 mg/mL, sufentanil citrate (Sufenta®) at 50 μg/mL; morphine sulfate at 20 mg/mL; fentanyl citrate (Sublimaze®) at 20 μg/ml and alfentanil hydrochloride at 500 μg/mL (see generally Physician's Desk Reference, Thomson Healthcare, Montvale, N.J., (2001) pp. 821, 826, 828, 830, 831, 1193 and 1619). U.S. Pat. No. 6,113,937 describes a sufentanil formulation suitable for intramuscular administration composed of a carboxylic acid of between 8 and 22 carbon atoms (such as stearic acid) combined with sufentanil in a 1:1 ratio, with a medium-chain triglyceride. However, the concentration of sufentanil disclosed in the formulations range between 0.1 and 1 mg/mL.

For the foregoing reasons, it is evident that there is a need in the art for more concentrated opioid and opioid derivative formulations that permit convenient long-term or high dose delivery, yet are physically and chemically stable over time and safe for parenteral use. The present invention addresses this need, and provides related advantages as well.

### LITERATURE

U.S. Pat. No. 6,113,937 describes a sufentanil formulation suitable for intramuscular administration composed of a carboxylic acid of between 8 and 22 carbon atoms (such as stearic acid) combined with sufentanil in a 1:1 ratio, with a medium-chain triglyceride. The concentration of sufentanil disclosed in the formulations range between 0.1 and 1 mg/mL.

Fudin et al. Am J Hospice Pallitave Care 2000 17:347-353 describes hydromorphone formulations in dextrose 5% in water at hydropmorphone concentrations of 10 mg/ml to 100 mg/ml, as well as in 0.9% normal saline at hydromorphone concentrations of 10 mg/ml to 100 mg/ml.

Wagner et al. Can Pharmakinet 1997 33:426-53 describe the pharmacokinetics and pharmacodynamics of sedatives and analgesics in the treatment of agitated critically ill patients.

Willens et al. Heart Lung 1993 22:239-52 describes pharmacodynamics, pharmacokinetics, and clinical uses of fentanyl, sufentanil, and alfentanil.

### SUMMARY OF THE INVENTION

The present invention provides opioid formulations suitable for long-term delivery to a subject. The formulation of the invention comprises an opioid or opioid derivative (e.g., morphine, hydromorphone, fentanyl or a fentanyl congener), and an aqueous solvent comprising a carboxylic acid, particularly a low molecular weight carboxylic acid (e.g., C₂₋₇, C₂₋₄. The invention thus provides for formulations comprising morphine, hydromorphone, fentanyl or fentanyl congeners in concentrations significantly in excess of conventional aqueous formulations, e.g., on the order about 2-fold to about 10,000-fold greater than conventional formulations, e.g., currently commercially available formulations.

The high concentrations of the formulations of the invention are especially useful for high-dose delivery, or long-term delivery, e.g., from a reservoir of a drug delivery device for a period of, e.g., several hours, weeks, months, or even years. Long-term delivery can be achieved using various external or implanted devices. The formulations of the invention are generally flowable at ambient (e.g., room) temperature, body temperature, or both ambient and body temperatures.

The invention further provides a sustained release dosage form comprising a formulation of the invention. The dosage form can be, for example, an external, partially implanted, or wholly (completely) implanted device (e.g., implants or pumps), which can be based on, for example, drug diffusion systems, pumps (e.g., electromechanical pumps, electrochemical pumps, osmotic pumps, vapor pressure pumps, electrolytic pumps, effervescent pumps, piezoelectric pumps, and the like), electrodiffusion systems, electroosmosis systems, and the like. In one embodiment, the sustained release dosage form is a controlled release dosage form.

The invention further provides methods of treating pain in a subject, comprising delivering from a drug delivery device an opioid or opioid derivative (e.g., morphine, hydromorphone, oxycodone, fentanyl or fentanyl congener) formulation of the invention to a subject in need of pain relief or prevention. Delivery of the formulation is generally continuous over a pre-selected administration period ranging from several hours, one to several weeks, one to several months, up to one or more years.

A primary advantage of the present invention is that very potent and concentrated opioid formulations can be achieved by solubilizing the drug in a small volume of an aqueous carboxylic acid solvent. The formulations of the invention are of particular use where the delivery device is relatively small (e.g., an implantable system), where delivery is required for a relatively long duration, or where high effective doses of drug are required to achieve the desired therapeutic effect. Thus, it is possible to deliver a consistent amount of drug over an extended period of time (e.g., days, weeks, months, etc.) without the need to refill or replace the delivery device, thereby reducing risk of infection and tissue damage, increasing patient compliance, and achieving consistent, accurate dosing.

Another advantage of the formulations of the invention is that high concentrations of opioid or opioid derivative (e.g., fentanyl or fentanyl congener) are achieved without substantial precipitation of the drug.

Another important advantage of the formulations of the present invention is that therapeutic amounts of drug (even high doses) can be delivered to a subject by using only very small volumes of formulation (e.g., on the order of microliters per day or nanoliters per day). In certain body tissues, e.g., subcutaneous space, low volume delivery facilitates better absorption of the drug by the local tissue, and minimizes local tissue disturbance, trauma, or edema.

These and other objects, advantages, and features of the invention will become apparent to those persons skilled in the art upon reading the details of the invention as more fully described below.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a formulation” includes a plurality of such formulations and reference to “the fentanyl congener” includes reference to one or more fentanyl congeners and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### DETAILED DESCRIPTION OF THE INVENTION

### Definitions

The terms “drug” and “therapeutic agent,” used interchangeably herein, are generally meant to refer to opioids and opioid derivatives, particularly morphine, hydromorphone, fentanyl or a fentanyl congener (e.g., sufentanil, alfentanil, lofentanil, carfentanil, remifentanil, trefentanil, and mirfentanil), as well as formulations comprising one or more of these compounds. Use of “drug” or, for example, the phrase “fentanyl or fentanyl congener” is not meant to be limiting to use of, or formulations comprising, only one of these selected opioid compounds.

The terms “opioid” and “opioid derivative” are used to refer to compounds related to morphine, and encompasses both natural (codeine, morphine) and synthetic (fentanyl, sufentanil) compounds. “Opioid derivative” also applies to agonists and antagonists with morphine-like activity.

The term “congener” as in “fentanyl congener” generally refers to a chemical compound that is related to another compound, such as a derivative the compound. For example, “fentanyl congeners” include derivatives of fentanyl such as sufentanil, alfentanil, carfentanil, and the like.

As used herein, and unless otherwise specified, the term “carboxylic acid” refers to any suitable carboxylic acid, usually a monocarboxylic acid, dicarboxylic acid, or tricarboxylic acid, more usually a monocarboxylic acid or dicarboxylid acid, normally a monocarboxlyic acid. A “low molecular weight carboxylyic acid” is meant to refer to a carboxylic acid having less than 8 carbon atoms, less than 7 carbon atoms, less than 6 carbon atoms, less than 5 carbon atoms, usually from about 2 to 7 carbon atoms, from about 2 to 6 carbon atoms, from about 2 to 5 carbon atoms, normally from about 2 to 4 carbon atoms.

“Pharmaceutically acceptable salt” and “salts thereof” in the compounds of the present invention refers to acid addition salts and base addition salts.

The term “carrier” as used in the present invention means a substantially inert material used as a vehicle for the drug.

The term “solvent” as used in the present invention encompasses a flowable composition, usually a liquid, which solubilizes the drug, e.g., so as to prevent precipitation. A solvent may also, in some instances, act as a carrier. A flowable solvent includes solvents that are flowable at ambient (e.g., room) temperature, body temperature, or both.

The term “formulation” as used in the present invention refers to a composition having a drug as a component. For example, an exemplary formulation of the invention comprises sufentantil, acetic acid and water.

The term “subject” is meant any subject, generally a mammal (e.g., human, primate, canine, feline, equine, bovine, etc.), in which management of pain is desired.

The term “therapeutically effective amount” is meant an amount of a therapeutic agent, or a rate of delivery of a therapeutic agent, effective to facilitate a desired therapeutic effect. The precise desired therapeutic effect (e.g., the degree of pain relief, and source of the pain relieved, etc.) will vary according to the condition to be treated, the formulation to be administered, and a variety of other factors that are appreciated by those of ordinary skill in the art. In general, the method of the invention involves the suppression or mitigation of pain in a subject suffering from pain that may be associated with any of a variety of identifiable or unidentifiable etiologies.

The term “pain management” is used here to generally describe regression, suppression, or mitigation of pain, including acute and chronic pain, so as to make the subject more comfortable as determined by subjective criteria, objective criteria, or both. In general, pain is assessed subjectively by patient report, with the health professional taking into consideration the patient's age, cultural background, environment, and other psychologic background factors known to alter a person's subjective reaction to pain.

“Delivery site” as used herein is meant to refer to an area of the body to which the drug is delivered. Such delivery sites include, but are not necessarily limited to, intravenous, intraspinal (e.g., epidural, subdural, or intrathecal), intracerebral, transdermal, intra-lymphatic, intra-lipose (e.g., within fatty tissue), intradermal, transdermal, or subcutaneous sites of delivery and the like. Exemplary subcutaneous delivery sites include external subcutaneous sites (e.g., under the skin of the arm, shoulder, neck, back, or leg) and internal subcutaneous sites within a body cavity (e.g., within the mouth).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug release device” or “controlled release dosage form” is meant to encompass any device wherein the release rate (e.g., rate of timing of release) of a drug or other desired substance contained therein is controlled by the device or dosage form itself and substantially not the environment of use, and that can be adapted for use in the invention, e.g., a dosage form that provides for controlled release of drug and at a rate that is suitable to accomplish delivery of a therapeutically effective amount of drug to a site within the body. The terms “device” and “dosage form” are generally used interchangeably herein.

The term “sustained release dosage form” is meant to refer to a drug dosage form that is adapted for release of a drug formulation (e.g., an opioid) over a pre-selected period of time rather than at one time as in a bolus administration (e.g., by injection or oral administration). Sustained release dosage forms can include dosage forms capable of controlled release or patterned release of a drug.

A “dosage form adapted for implantation” is meant to refer to any dosage form that is suitable for introduction and retention in a site within a subject, generally a parenteral site within a subject (e.g., subcutaneous site, intramuscular site, and the like).

“Treatment” as in “treatment of pain” is used herein to encompass both a decrease in pain severity and/or intensity to provide partial or complete relief of pain and/or pain symptoms. The effect may be prophylactic in terms of completely or partially preventing or reducing the severity of pain.

### Overview

The present invention is based on the finding that opioids and opioid derivatives, such as morphine, hydromorphone, fentanyl and its congeners, can be formulated at high concentrations when an aqueous solvent comprising a low molecular weight carboxylic acid is used.

The use of carboxylic acids as solvents to create high concentration opioid preparations is an unexpected result because in general, the opioids have relatively low solubility in aqueous formulations. Because of this low solubility issue, previous attempts at increasing the concentration of opioid formulations have focused on non-aqueous formulations, particularly alcohol solvents. The formulations of the present invention comprise an opioid prepared with a carboxylic acid and water to give a final concentration of up to about 400-600 mg/mL or more, or, stated differently, having a molar ratio of carboxylic acid to drug of greater than 0.5 to 1.5, depending on the drug used and the formulation.

The present invention provides formulations of any desirable opioid or opioid derivative, with hydromorphone, fentanyl and fentanyl congeners being of particular interest. These formulations are generally characterized in that: (1) they have a concentration of about 2 to about 10,000 times greater than that of commercially available formulations; (2) the drug does not precipitate out when the formulation comes into contact with an aqueous environment, e.g., in the body of the subject being treated; and (3) have good stability, even at body temperatures.

The opioid or opioid derivatives can be provided in any of a variety of formulations compatible with parenteral delivery, provided that such formulation is stable (i.e., not subject to degradation to an unacceptable amount at body temperature). The concentration of the formulation may vary from about 0.1 wt. % to about 50 or 75 wt. %. The drug can be provided in any form suitable to be carried by the drug delivery device and released parenterally for systemic distribution, and is generally a flowable formulation, e.g., gel, liquid, suspension, emulsion, etc., at ambient (e.g., room) temperature, at body temperature, or at both ambient room and body temperature.

The morphine, hydromorphone, fentanyl or fentanyl congener is generally soluble in the formulation, i.e., little or no precipitates are present, and further, little or no drug precipitates when the formulation comes in contact with an aqueous environment such as a body fluid. Precipitates of morphine, hydromorphone, fentanyl or fentanyl congeners, when present at all, are present in the formulation at less than about 10%, less than about 7.5%, less than about 5%, less than about 2.5%, less than about 1%, or less than about 0.1% by weight of the total drug in the formulation. Whether precipitates have formed can be determined using any method known in the art, including, but not limited to, visual inspection with the unaided eye, or under low (e.g., 10× or 25×) magnification.

The formulations useful in the invention can comprise inactive ingredients and/or other active ingredients (e.g., in addition to the opioid or opioid derivative).

### Carboxylic Acid Solvents

Pharmaceutical grade organic or inorganic carriers and/or diluents suitable for systemic delivery can be included in the formulation suitable for delivery according to the invention. Such physiologically acceptable carriers are well known in the art. Exemplary liquid carriers for use in accordance with the present invention are sterile aqueous solutions, which contain water plus a carboxylic acid such as acetic acid, lactic acid, or salt thereof. Suitable aqueous carriers may optionally further comprise more than one buffer salt, as well as other salts (such as sodium and potassium chlorides) and/or other solutes.

In a preferred embodiment, the formulation comprises water and a carboxylic acid, such as acetic acid or lactic acid, or salts thereof and/or mixtures or admixtures thereof. Carboxylic acids suitable for use include carboxylic acids having 2, 3, 4, 5, 6, or 7 carbon atoms, usually from 2 to 4 carbon atoms, including mono-, di-, and tri-carboxylic acids, usually mono- or di-carboxylic acids, where the compound can comprise an α, β, and/or γ hydroxyl, alkyl, or alkylhydroxy group.

Carboxylic acids of particular interest for use in production of fentanyl/fentanyl congener formulations include C1-C3 carboxylic acids and α-hydroxyl, β-hydroxyl and/or γ-hydroxyl derivatives thereof. Specific exemplary acids usable with the invention include, but are not necessarily limited to, the following:

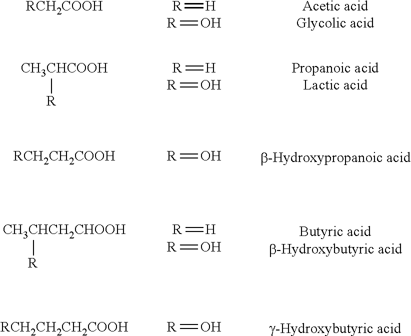

and salts, mixtures and admixtures thereof. Where available, the carboxylic acids may be in any isomeric form, e.g., the D- or L-stereoisomer. The carboxylic acid solvents used in the invention may be present in a molar concentration about equal to or greater than that of the drug (e.g., present in a molar ratio of 1:1 (i.e., 1) or 2:1 (i.e., 2) carboxylic acid to drug). For example, the molar ratio of carboxylic acid to drug in the formulation can be about 1, greater than about 0.5, greater than about 1, about 1.5, about 1.8, about 2, about 2.2, about 2.5, about 2.8, about 3, about 3.5, or more.

Such carboxylic acid-containing formulations allow for a very high drug concentration. This high concentration provides for longer duration of drug delivery, and provides excellent chemical stability for the sufentanil formulation. For example, a morphine, hydromorphone, fentanyl or a fentanyl congener in a carboxylic acid formulation comprising lactate may have a concentration of at least 600 mg/ml, for example about 100 to 600 mg/ml, or for example about 300 mg/ml to 600 mg/ml, or about 500 to 600 mg/ml. Concentrations of morphine, hydromorphone, fentanyl or a fentanyl congener in a carboxylic acid formulation comprising acetate may be at least about 400 mg/ml, for example 100 mg/ml to 400 mg/ml, or for example 300 mg/ml to 400 mg/ml.

The drug in the formulation can be the free base or any suitable pharmaceutically acceptable salt of the drug. For example, the drug can be the acetate salt, lactate, salt, citrate salt, and other carboxylic acid salts. In general the drug is either the free base form or is a drug salt compatible with the selected carboxylic acid, e.g., where the carboxylic acid solvent is lactic acid, the drug salt can be the lactate salt.

Exemplary formulations are described in more detail below.

### Fentanyl or Fentanyl Congener Formulations

The invention provides a formulation, particularly a pharmaceutical formulation, comprising fentanyl or a fentanyl congener.

Formulations of the invention comprise fentanyl or a fentanyl congener in a concentration of at least about 0.1 mg/mL, 1 mg/mL, 5 mg/mL, 10 mg/mL, 25 mg/mL, 50 mg/mL, 75 mg/mL, 100 mg/mL, 150 mg/mL, 200 mg/mL, 225 mg/mL, 250 mg/mL, 300 mg/mL, 350 mg/mL, 400 mg/mL, 450 mg/mL, 500 mg/mL, and may be up to about 600 mg/ml, or greater. Formulations of the invention comprising fentanyl or fentanyl congener are in aqueous solution, e.g., are dissolved in a formulation comprising water.

The formulations of the invention allow for very high drug concentration, thus allowing longer duration of drug delivery, and provide excellent chemical stability for the formulations (with the acetate and lactate salts being of particular interest). For example, concentration of drug in a formulation of the invention may be up to about 600 mg/ml, for example about 100 to 600 mg/ml, or for example about 300 mg/rat to 600 mg/ml, or about 400 to 600 mg/ml. Concentration of fentanyl or a fentanyl congener in an aqueous formulation comprising acetate may be up to about 400 mg/ml, for example 100 mg/ml to 400 mg/ml, or for example 300 mg/ml to 400 mg/ml.

The fentanyl or fentanyl congener is present in the formulation in a concentration substantially higher than conventional formulations, e.g., current commercially available formulations. By “substantially higher,” it is intended that the fentanyl or fentanyl congener is present in the formulation in a concentration of at least about 2, at least about 5, at least about 10, at least about 20, at least about 50, at least about 100, at least about 250, at least about 500, at least about 1000, at least about 1500, at least about 2000, at least about 2500, at least about 3000, at least about 3500, at least about 4000, at least about 5000, at least about 6000, at least about 7000, at least about 8000, at least about 9000, at least about 10,000 times, or greater, than the solubility of fentanyl or fentanyl congener that is commercially available.

Fentanyl, congeners of fentanyl, and specific derivatives or analogs of fentanyl or fentanyl congeners (e.g., other derivatives, particularly 4-anilidopiperidine derivatives, of morphine) are contemplated for delivery according to the invention, although variations within the scope of the invention will be readily apparent to the ordinarily skilled artisan upon reading the disclosure provided herein. Exemplary fentanyl congeners include, but are not necessarily limited to sufentanil, alfentanil, lofentanil, carfentanil, remifentanil, trefentanil, and mirfentanil.

The specific fentanyl congener used can vary with a variety of factors, including the therapeutic effect desired to be achieved, the patient's tolerance and/or previous exposure to opioids, etc. The relative potency of fentanyl or the fentanyl congener may also be considered in selection of the drug to be delivered. For example, the rank order of potency of fentanyl and selected fentanyl congeners relative to morphine is as follows: morphine<alfentanil<fentanyl<sufentanil<lofentanil<carfentanil. For a review of the pharmacokinetics of sufentanil, fentanyl, and other fentanyl congeners, see, e.g., Meert (1996) Pharm. World Sci. 18:1-15; and Scholz et al. (1996) Clin. Pharmacokinet. 31:275-92.

Methods for manufacture of fentanyl, sufentanil and other fentanyl congeners are well known in the art, see, e.g., sufentanil (e.g., U.S. Pat. No. 3,998,834; chemical name: ((N-[4-(methyoxymethyl)-1-[2-(2-thienyl)ethyl]-4-piperidinyl]-N-phenylpropanamide 2-hydroxy-1,2,3,-propanetricarboxylate (1:1); C₂₂H₃₀N₂O₂S), fentanyl (e.g., U.S. Pat. No. 3,141,823; chemical name: N-phenyl-N-[1-(2-phenylethyl)-4-piperidinyl]propanamide), alfentanil (e.g., U.S. Pat. No. 4,167,574; chemical name: N-[1-[2-(4-ethyl-4,5-dihydro-5-oxo-1H-tetrazol-1-yl)ethyl]-4-(methoxymethyl)-4-piperidinyl]-phenylpropanamide (C₂₁H₃₂N₆O₃)), lofentanil (e.g., U.S. Pat. No. 3,998,834; chemical name: 3-methyl-4-[(1-oxopropyl)phenylamino]-1-(2-phenylethyl)-4-piperidinecarboxylic acid methyl ester), carfentanil (chemical name: methyl-4-[(1-oxopropyl)phenylamino]-1-(2-phenylethyl)-4-piperidinecarboxylate (C₂₄H₃₀N₂O₃)), remifentanil (chemical name: 3-[4-methoxycarbonyl-4-[(1-oxopropyl) phenylamino]1-piperidine]propanoic acid), trefentanil (chemical name: N-(1-(2-(4-ethyl-4,5-dihydro-5-oxo-1H-tetrazol-1-yl)ethyl)-4-phenyl-4-piperidinyl)-N-(2-fluorophenyl)-propanamide, and mirfentanil (chemical name: [N-(2-pyrazinyl)-N-(1-phenethyl-4-piperidinyl)-2-furamide).

Fentanyl and fentanyl congeners are discussed in detail in, for example, Goodman and Gilman's The Pharmacological Basis of Therapeutics, Chapter 23, “Opioid Analgesics and Antagonists” pp. 521-555 (9^{th} Ed. 1996); Baly et al. (1991) Med. Res. Rev. 11:403-36 (evolution of the 4-anilidopiperidine opioids); and Feldman et al. (1991) J. Med. Chem. 34:2202-8 (design, synthesis, and pharmacological evaluation of opioid analgesics). For additional information on fentanyl and fentanyl congeners, see, e.g., Scholz et al. (1996) Clin. Pharmacokinet. 31:275-92 (clinical pharmacokinetics of alfentanil, fentanyl, and sufentanil); Meert (1996) Pharmacy World Sci. 18:1-15 (describing pharmacotherapy of morphine, fentanyl, and fentanyl congeners); Lemmens et al. (1995) Anesth. Analg. 80:1206-11 (pharmacokinetics of mirfentanil); Minto et al., (1997) Int. Anesthesial. Clin. 35:49-65 (review of recently developed opioid analgesics); James (1994) Expert Opin. Invest. Drugs 3:331-40 (discussion of remifentanil); Rosow (1993) Anesthesiology 79:875-6 (discussion of remifentanil); Glass (1995) Eur. J. Anaesthesiol. Suppl. 10:73-4 (pharmacology of remifentanil); and Lemmens et al. (1994) Clin. Pharmacal. Ther. 56:261-71 (pharmacokinetics of trefentanil).

Fentanyl or a fentanyl congener can be provided in the formulation as the opioid base and/or the opioid pharmaceutically acceptable salt, but is preferably provided in the formulation as the opioid base. The pharmaceutically acceptable salt embraces the inorganic and the organic salt. Representative salts include a member selected from the group consisting of hydrobromide, hydrochloride, mutate, citrate, succinate, n-oxide, sulfate, malonate, acetate, phosphate dibasic, phosphate monobasic, acetate trihydrate, bi(heptafluorobutyrate), maleate, bi(methylcarbamate), bi(pentafluoropropionate), mesylate, bi(pyridine-3-carboxylate), bi(trifluoroacetate), bitartrate, chlorhydrate, fumarate and sulfate pentahydrate. Where the drug formulation comprises sufentanil, use of the sufentanil base is specifically contemplated for use.

### Hydromorphone Formulations

The invention provides formulations, particularly a pharmaceutical formulation, comprising hydromorphone.

Formulations of the invention comprise hydromorphone in a concentration of at least about 20 mg/mL, 50 mg/mL, 75 mg/mL, 100 mg/mL, 150 mg/mL, 200 mg/mL, 225 mg/mL, 250 mg/mL, 300 mg/mL, 350 mg/mL, 400 mg/mL, 450 mg/mL, 500 mg/mL, and may be up to about 600 mg/mL or greater. Formulations of the invention comprising hydromorphone are in aqueous solution, e.g., are dissolved in a formulation comprising water.

Hydromorphone hydrochloride (Dilaudid™) is a hydrogenated ketone of morphine, and is an ideal opioid for use by the subcutaneous route due to its efficacy and potency. Commercial preparations of high potency Dilaudid™ have a concentration of 10 mg/mL in sterile water for injection (Physician's Desk Reference, pp. 1619-1621 (2001)). The carboxylic acid formulations of the present invention, by comparison, have a hydromorphone concentration of about 600 mg/mL. This allows the same volume of liquid infused into a patient to be given for a longer period of time. The long-term stability of the hydromorphone formulations has been confirmed for a period of 28 days (Fudin, J. et al. (2000) Am. J. Hosp. Pall. Care 17(5):347-353). Hydromorphone solution is subject to oxidation over time. The rate of decomposition is pH and oxygen-dependent, that is, the decomposition is faster at a pH above 5. However, this decomposition rate can vary depending on the formulation.

The hydromorphone is present in the formulation in a concentration substantially higher than conventional formulations, e.g., current commercially available formulations. By “substantially higher,” it is intended that the hydromorphone is present in the claimed formulation in a concentration of at least about 2, at least about 5, at least about 10, at least about 20, at least about 35, at least about 50, at least about 100, at least about 250, at least about 500, at least about 1000, at least about 1500, at least about 2000, at least about 2500, at least about 3000, at least about 3500, at least about 4000, at least about 5000, at least about 6000, at least about 7000, at least about 8000, at least about 9000, at least about 10,000 times, or greater, than the solubility of hydromorphone in commercially available solution.

### Dosage Forms Useful in the Methods of the Invention

Any of a variety of dosage forms can be used in conjunction with a formulation of the present invention. Delivery methods and dosage forms suitable for use with the formulations of the present invention can take advantage of any of a variety of drug release mechanisms.

In general, the dosage forms suitable of interest for use with the formulations of the invention are adapted for retaining a quantity of drug formulation (e.g., contained in a drug reservoir or solubilized, suspended or integrated into a vehicle, substrate or matrix such as a polymer, binding solid, etc.) sufficient for treatment for a pre-selected period. In general the dosage forms for use with the present invention are adapted for sustained release of the formulation. Exemplary dosage forms include drug delivery devices (e.g., drug pumps), transdermal delivery devices, bioerodable implants, sustained release injectables (e.g., injectable high viscous formulations, gels including hydrogels such as collagen hydrogels), microparticulate suspensions, microsphere suspensions, liposome formulations, micelle formulations, oil suspensions (including emulsions), and encapsulated particulate suspensions. Drug delivery dosage forms that may be suitable for use with the present invention are described in Encyclopedia of Controlled Drug Delivery (1999), E. Mathiowitz (Ed.), John Wiley & Sons, Inc. The dosage form can be selected from, for example, any of a variety of conventional drug release devices that are conventionally used as an external element (e.g., an external pump) or implanted element of a drug delivery system.

In some embodiments, the dosage form (also referred to herein as a delivery device) is one that is adapted for delivery of drug over an extended period of time. Such delivery devices may be adapted for administration of fentanyl or fentanyl congener for several hours (e.g., 2 hours, 12 hours, or 24 hours to 48 hours or more), to several days (e.g., 2 to 5 days or more, from about 100 days or more), to several months or years. In some of these embodiments, the device is adapted for delivery for a period ranging from about 1 month to about 12 months or more. The drug delivery device may be one that is adapted to administer fentanyl or fentanyl congener to an individual for a period of, e.g., from about 2 hours to about 72 hours, from about 4 hours to about 36 hours, from about 12 hours to about 24 hours; from about 2 days to about 30 days, from about 5 days to about 20 days, from about 7 days to about 100 days or more, from about 10 days to about 50 days; from about 1 week to about 4 weeks; from about 1 month to about 24 months or more, from about 2 months to about 12 months, from about 3 months to about 9 months; or other ranges of time, including incremental ranges, within these ranges, as needed.

Release of drug from the dosage form, particularly sustained or controlled release of drug, can be accomplished in any of a variety of ways according to methods well known in the art, e.g., by solubilization or suspension of drug in a vehicle or incorporation of drug into a polymer that provides for substantially controlled diffusion of drug from within the polymer, incorporation of drug in a biodegradable polymer, providing for delivery of drug from an osmotically-driven device, etc. Where the drug delivery device comprises a drug delivery catheter, drug can be delivered through the drug delivery catheter to the delivery site as a result of capillary action, as a result of pressure generated from the drug device, by diffusion, by electrodiffusion or by electroosmosis through the device and/or the catheter.

In general, the dosage form is adapted to carry the drug formulation in such quantities and concentration as therapeutically required for treatment over the pre-selected period, and must provide sufficient protection to the formulation from degradation by body processes for the duration of treatment. For example, the dosage form can be surrounded by an exterior made of a material that has properties to protect against degradation from metabolic processes and the risk of, e.g., leakage, cracking, breakage, or distortion. This can prevent expelling of the dosage form contents in an uncontrolled manner under stresses it would be subjected to during use, e.g., due to physical forces exerted upon the drug release device as a result of movement by the subject or for example, in convective drug delivery devices, physical forces associated with pressure generated within the reservoir. The drug reservoir or other means for holding or containing the drug must also be of such material as to avoid unintended reactions with the active agent formulation, and is preferably biocompatible (e.g., where the dosage form is implanted, it is substantially non-reactive with respect to a subject's body or body fluids).

Drug release devices suitable for use in the invention may be based on any of a variety of modes of operation. For example, the drug release device can be based upon a diffusive system, a pump system, or an erodible system. Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,2440; 4,203,442; 4,210,139; 4,327,725; 4,627,850; 4,865,845; 5,057,318; 5,059,423; 5,112,614; 5,137,727; 5,234,692; 5,234,693; 5,728,396; 5,985,305; and the like.

In one embodiment, the drug release device is a controlled drug release device in the form of an osmotically-driven device. Preferred osmotically-driven drug release systems are those that can provide for release of agent in a range of rates of from about 0.01 mg/hr to about 1000 mg/hr, and which can be delivered at a volume rate range of, for example, from about 0.001 ml/day to about 100 ml/day (i.e., from about 0.0004 ml/hr to about 4 ml/hr), from about 0.04 ml/day to about 10 ml/day, from about 0.2 ml/day to about 5 ml/day, from about 0.5 ml/day to about 1 ml/day. In general, in the present invention, the drug release system is selected to provide for delivery of drug at a rate of from about 0.001 ml/day (1 ml/day) to at least about 500 ml/day or about 1 ml/day (i.e., from about 0.04 ml/hr to about 21 ml/hr to about 42 ml/hr), from about 2 ml/day to about 250 ml/day to 500 ml/day, from about 4 ml/day to about 100 ml/day, from about 5 ml/day to about 50 ml/day to 250 ml/day.

In an embodiment, the sustained release dosage form is a depot-type injectable, see e.g., U.S. Pat. Nos. 6,183,781; 6,174,547; 6,156,331; 6,143,314; 6,130,200; 6,120,789; 6,051,558; 5,989,463; 5,968,542; 5,912,015; 5,747,058; 5,702,716; 5,654,008; and 5,650,173.

In one embodiment of particular interest, the volume/time delivery rate is substantially constant (e.g., delivery is generally at a rate±about 5% to 10% of the cited volume over the cited time period). In one embodiment, the drug release device is a continuous drug release device in the form of an osmotically-driven device. Preferred osmotically-driven drug release systems are those that can provide for release of drug in a range of rates of from about 0.1 mg/hr to about 1000 mg/hr, and which can be delivered at a volume rate of from about 0.25 ml/day to about 100 ml/day (i.e., from about 0.0004 ml/hr to about 4 ml/hr), from about 0.04 ml/day to about 10 ml/day, and can be from about 0.2 ml/day to about 5 ml/day, or from about 0.5 ml/day to about 1 ml/day. In one embodiment, the volume/time delivery rate is substantially constant (e.g., delivery is generally at a rate±about 5% to 10% of the cited volume over the cited time period).

The invention features methods for management of pain by delivery of a formulation of the invention. In one embodiment, the drug formulation of the invention is delivered in a substantially continuous fashion. While the formulations of the invention can be delivered to any of a variety of delivery sites, the formulations can find particular use in delivery of hydromorphone, fentanyl or a fentanyl congener to a site at or under the skin, with a subcutaneous or intradermal site being of particular interest.

### Pain Susceptible to Management According to the Methods of the Invention

In general, administration of a formulation of the invention can be used to facilitate management of pain that is associated with any of a wide variety of disorders, conditions, or diseases. Causes of pain may be identifiable or unidentifiable. Where identifiable, the origin of pain may be, for example, of malignant, non-malignant, infectious, non-infectious, or autoimmune origin. Of particular interest is the management of pain associated with disorders, diseases, or conditions that require long-term therapy, e.g., chronic and/or persistent diseases or conditions for which therapy involves treatment over a period of several days (e.g., about 3 days to 10 days), to several weeks (e.g., about 3 or 4 weeks to 6 weeks), to several months or years, up to including the remaining lifetime of the subject. Subjects who are not presently suffering from a disease or condition, but who are susceptible to such may also benefit from prophylactic pain management using the devices and methods of the invention, e.g., prior to traumatic surgery. Pain amenable to therapy according to the invention may involve prolonged episodes of pain alternating with pain-free intervals, or substantially unremitting pain that varies in severity.

In general, pain can be somatogenic, neurogenic, or psychogenic. Somatogenic pain can be muscular or skeletal (i.e., osteoarthritis, lumbosacral back pain, posttraumatic, myofascial), visceral (i.e., chronic pancreatitis, ulcer, irritable bowel), ischemic (i.e., arteriosclerosis obliterans), or related to the progression of cancer (e.g., malignant or non-malignant). Neurogenic pain can be due to posttraumatic and postoperative neuralgia, can be related to neuropathies (i.e., diabetes, toxicity, etc.), and can be related to nerve entrapment, facial neuralgia, perineal neuralgia, postamputation, thalamic, causalgia, and reflex sympathetic dystrophy.

Specific examples of conditions, diseases, disorders, and origins of pain amenable to management according to the present invention include, but are not necessarily limited to, cancer pain (e.g., metastatic or non-metastatic cancer), chronic inflammatory disease pain, neuropathic pain, post-operative pain, iatrogenic pain (e.g., pain following invasive procedures or high dose radiation therapy, e.g., involving scar tissue formation resulting in a debilitating compromise of freedom of motion and substantial chronic pain), complex regional pain syndromes, failed-back pain (chronic back pain), soft tissue pain, joints and bone pain, central pain, injury (e.g., debilitating injuries, e.g., paraplegia, quadriplegia, etc., as well as non-debilitating injury (e.g., to back, neck, spine, joints, legs, arms, bands, feet, etc), arthritic pain (e.g., rheumatoid arthritis, osteoarthritis, arthritic symptoms of unknown etiology, etc.), hereditary disease (e.g., sickle cell anemia), infectious disease and resulting syndromes (e.g., Lyme disease, AIDS, etc.), chronic headaches (e.g., migraines), causalgia, hyperesthesia, sympathetic dystrophy, phantom limb syndrome, denervation, and the like. Pain can be associated with any portion(s) of the body, e.g., the musculoskeletal system, visceral organs, skin, nervous system, etc.

Cancer pain is an example of one broad category of pain that can be alleviated according to the methods of the invention. One of the underlying causes of cancer pain is the severe local stretching of tissues by the neoplastic lesion. For example, as the cancer cells proliferate in an unrestricted manner, the tissues in the local region of cancer cell proliferation are subjected to mechanical stress required to displace tissue and accommodate the increased volume occupied by the tumor mass. When the tumor burden is confined to a small, enclosed compartment, such as the marrow of a bone, the resulting pressure can result in severe pain. Another cause of pain can result from the aggressive therapies used to combat the patient's cancer, e.g., radiation therapy, chemotherapy, etc. Such cancer therapies can involve localized or widespread tissue damage, resulting in pain.

Pain associated with any type of malignant or non-malignant cancer is amenable to alleviation according to the invention. Specific examples of cancers that can be associated with pain (due to the nature of the cancer itself or therapy to treat the cancer) include, but are not necessarily limited to lung cancer, bladder cancer, melanoma, bone cancer, multiple myeloma, brain cancer, non-Hodgkin's lymphoma, breast cancer, oral cancers, cervical cancer, ovarian cancer, colon cancer, rectal cancer, pancreatic cancer, dysplastic nevi, endocrine cancer, prostate cancer, head and neck cancers, sarcoma, Hodgkin's disease, skin cancer, kidney cancer, stomach cancer, leukemia, testicular cancer, liver cancer, uterine cancer, and aplastic anemia. Certain types of neuropathic pain can also be amenable to treatment according to the invention.

Chronic back pain, which is also amenable to management using the methods of the invention, is another broad category of pain that can be alleviated by application of the methods of the invention. Chronic back pain is generally due to one or more of the following six causes: (i) stress on intervertebral facet joints, caused by slippage, arthritis, wedging, or scoliosis; (ii) radiculopathy, the mechanical compression of the nerve root due to bulging discs or tumors; (iii) tendonitis or tendon sprain; (iv) muscle spasm or muscle sprain; (v) ischemia, a local insufficiency in circulatory flow; and (vi) neuropathy, damage to nervous tissue of metabolic etiology or arising from cord tumors or central nervous system disease.

The methods of the invention can be used to manage pain in patients who are opioid naive or who are no longer opioid naive. Exemplary opioid naive patients are those who have not received long-term opioid therapy for pain management. Exemplary non-opioid naive patients are those who have received short-term or long-term opioid therapy and have developed tolerance, dependence, or other undesirable side effects. For example, patients who have intractable adverse side effects with oral, intravenous, or intrathecal morphine, transdermal fentanyl patches, or other conventional methods and devices of opioid delivery can achieve good analgesia and maintain favorable side-effects profiles with delivery of fentanyl or a fentanyl congener when administered in the dose ranges and/or low volume rates described above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Sufentanil Formulation in Lactic Acid Solvent

A formulation with a concentration of 600 mg/ml sufentanil was made by adding approximately 30 g of sufentanil to 14 ml of 88% L-lactic acid, followed by 15 ml of water, and stirring the mixture until all the solute was dissolved. Water was added to the resulting solution to give a total volume of 50 ml. The molar ratio of L-lactic acid to drug in the final formulation was approximately 2, with a final pH of 4.5

### Example 2

### Sufentanil Formulation in Acetic Acid Solvent

A formulation of about 500 mg/ml sufentanil was prepared by adding approximately 1.03 g sufentanil free base to 0.7 mL mixture of 50:50 acetic acid:water, followed by 0.5 mL water. The mixture was stirred to dissolve. Water was added to the resulting solution to make a 2 mL volume. The molar ratio of acetic acid to drug was approximately 2.3, with a final pH of 4.6.

### Example 3

### Hydromorphone Formulation in Acetic Acid Solvents

A formulation of about 600 mg/ml hydromorphone was prepared by adding approximately 15 g hydromorphone base to 9 mL glacial acetic acid, followed by 0.5 mL water. The mixture was stirred to dissolve the drug. Water was added to the resulting solution to make a 25 mL volume. The molar ratio of acetic acid to drug was approximately 3, with a final pH of 4.6.

### Example 4

### Preparation of Hydromorphone Lactic Acid Formulation

A formulation of about 600 mg/ml hydromorphone was prepared by adding approximately 15 g hydromorphone base to 6.75 mL 85% L-lactic acid, followed by 1.5 mL water. The mixture was stirred to dissolve. Water was added to the resulting solution to make a 25 mL volume. The molar ratio of L-lactic acid to drug was approximately 1.5, with a final pH of 4.6.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A pharmaceutical formulation comprising:
an aqueous solvent comprising a low molecular weight carboxylic acid or salt thereof; and
a drug selected from fentanyl and sufentanil; wherein the drug is present in the formulation at a concentration of at least 100 mg/ml.

2. The pharmaceutical formulation of claim 1, where the carboxylic acid is acetic acid, lactic acid, or a salt thereof.

3. The pharmaceutical formulation of claim 1, where the drug is sufentanil.

4. The pharmaceutical formulation of claim 3, where the carboxylic acid is acetic acid, lactic acid, or a salt thereof.

5. The pharmaceutical formulation of claim 1, where the drug is fentanyl.

6.-7. (canceled)

8. The pharmaceutical formulation of claim 1, wherein the drug is present in the formulation at a concentration of at least 200 mg/mL.

9. The pharmaceutical formulation of claim 1, wherein the drug is present in the formulation at a concentration of at least 500 mg/mL.

10. A pharmaceutical formulation comprising:
an aqueous solvent comprising a low molecular weight carboxylic acid or salt thereof; and
a drug selected from fentanyl and sufentanil; wherein the molar ratio of carboxylic acid or carboxylic acid salt to drug in the formulation is greater than 1.

11. The pharmaceutical formulation of claim 10, where the carboxylic acid is acetic acid, lactic acid, or a salt thereof.

12. The pharmaceutical formulation of claim 10, where the drug is sufentanil.

13. The pharmaceutical formulation of claim 12, where the carboxylic acid is acetic acid, lactic acid, or a salt thereof.

14. The pharmaceutical formulation of claim 10, where the drug is fentanyl

15. (canceled)

16. The pharmaceutical formulation of claim 10, wherein the molar ratio of carboxylic acid or carboxylic acid salt to drug in the formulation is greater than 2.

17. The pharmaceutical formulation of claim 10, wherein the molar ratio of carboxylic acid or carboxylic acid salt to drug in the formulation is greater than 2.5.

18. A pharmaceutical formulation comprising:
an aqueous solvent comprising a low molecular weight carboxylic acid or salt thereof; and
an opioid or opioid derivative; wherein the opioid or opioid derivative is present in the formulation at a concentration of at least 100 mg/ml.

19. The pharmaceutical formulation of claim 18, where the carboxylic acid is acetic acid, lactic acid, or a salt thereof.

20. The pharmaceutical formulation of claim 18, wherein the opioid or opioid derivative is selected from the group consisting of morphine, hydromorphone, fentanyl, and sufentanil.

21. The pharmaceutical formulation of claim 18, wherein the opioid or opioid derivative is hydromorphone.

22. The pharmaceutical formulation of claim 21, where the carboxylic acid is acetic acid, lactic acid, or a salt thereof.

23. (canceled)

24. The pharmaceutical formulation of claim 18, wherein the the opioid or opioid derivative is present in the formulation at a concentration of at least 500 mg/mL.

25. A dosage form adapted for sustained delivery of a drug to a subject, the dosage form comprising the pharmaceutical formulation according to claim 1.

26. (canceled)

27. A dosage form adapted for sustained delivery of a drug to a subject, the dosage form comprising the pharmaceutical formulation according to claim 10.

28. A dosage form adapted for sustained delivery of a drug to a subject, the dosage form comprising the pharmaceutical formulation according to claim 18.

29. A method of treating pain in a subject, comprising administering the pharmaceutical formulation according to claim 1 to the subject in an amount effective to alleviate pain in the subject.

30. A method of treating pain in a subject, comprising administering the pharmaceutical formulation according to claim 10 to the subject in an amount effective to alleviate pain in the subject.

31. A method of treating pain in a subject, comprising administering the pharmaceutical formulation according to claim 18 to the subject in an amount effective to alleviate pain in the subject.
